(19) United States
(12) Patent Application Publication
(10) Pub. No.: US 2023/0321167 A1
(43) Pub. Date: Oct. 12, 2023

(54) COMPOSITIONS AND METHODS FOR TREATMENT OF CANCERS

(71) Applicant: Apollon Formularies PLC, London (GB)

(72) Inventor: Stephen Douglas Barnhill, League City, TX (US)

(73) Assignee: Apollon Formularies PLC, London, ENG (GB)

(21) Appl. No.: 18/017,467

(22) PCT Filed: Aug. 1, 2021
(86) PCT No.: PCT/IB2021/057017

§ 371 Date: Jan. 23, 2023

Related U.S. Application Data
(60) Provisional application No. 63/054,337, filed on Jul. 21, 2020.

(51) Int. Cl. A61K 36/18; A61K 31/00; A61K 36/185; A61K 45/06; A61K 36/07; A61K 36/074

(52) U.S. Cl. CPC A61K 36/07; A61K 31/658; A61K 36/074; A61K 36/18; A61K 36/185; A61K 45/06

(57) ABSTRACT

Compositions and methods are provided for the treatment of a variety of cancers. The compositions generally comprise at least one edible or medicinal mushroom or extract thereof, at least one cannabinoid, at least one terpene, and at least one flavonoid. Methods for optimizing compositions using artificial intelligence algorithms are also provided.

### CROSS-REFERENCE TO RELATED APPLICATIONS

This claims the benefit of United States Provisional Patent Application No. 63/054,337 filed Jul. 21, 2020, the entirety of which is incorporated herein by reference.

### BACKGROUND

### Field of the Invention

This relates generally to compositions and methods useful in the treatment of cancers and for patients with cancer diagnoses. More particularly this relates to nutraceutical and/or pharmaceutical compositions comprising mushrooms or extracts thereof, cannabis extracts, terpenes, and flavonoids, and the like.

### Description of Related Art

Cancer remains one of the leading causes of death in the modern world. While some types have become more treatable over time, still other types resist treatment and are difficult to cure. Common types of cancer vary depending on the geography (i.e. the country or part of the world), dietary habits, environmental considerations, exposure to carcinogens, genetics, phenotype, and more.

Cancers are generally characterized by genetic changes that result in uncontrolled cell growth, lack of normal differentiation, failure to respond to apoptosis and other normal cell signals; promoting aberrant angiogenesis, and abnormal immune response to the cancer cells. The genetic changes are most frequently in connection with proto-oncogenes, tumor suppressor genes, and DNA repair genes.

Cancer treatment is difficult in many cases and depends on the type of cancer involved and how advanced it is/how far it has progressed. Some people with cancer have only one option presented for medical treatment, while others may have multiple treatment possibilities. Multiple options or a combination of available treatments may be presented to other patients depending on their own health status and genetics, the type and stage of the cancer at issue (including its detailed genotype and phenotype), and their ability to withstand the various options physically, emotionally, and/or financially. Generally, possible cancer treatments include surgery, chemotherapy, radiation therapy, immunotherapy, targeted therapy, hormone therapy, and combinations of any of the foregoing.

The risks of each option are well-established and each leaves much to be desired. Surgery is not possible in many cases as the cancer could not be surgically removed safely, and many potential patients because of age, or health status are would not able to withstand the process of anesthesia, surgery, blood loss, risk of infection, healing, and long-term recovery. Chemotherapy generally involves the use of highly toxic substances that do not discriminate well (or at all) between cancer cells and normal cells that happen to be e.g., dividing. Radiation therapy utilizes ionizing radiation or the like to kill cancer cells but often impacts many healthy noncancerous cells, even in its most highly targeted forms. Both chemotherapy and radiation treatment carry serious side-effects that increase the stigma of a cancer diagnosis for patients including but not limited to hair loss, nausea, cachexia, loss of appetite, fatigue / loss of energy, neuropathy and other pain, inflammation, throat and/or mouth problems, sexual health problems, confusion and problems with concentration and memory, constipation and/or diarrhea, edema, and general malaise.

Other treatments such as immunotherapy, targeted therapy, and hormone therapy can be great but are limited in their application. If a particular cancer is responsive to an immunotherapeutic, e.g. a monoclonal antibody, a person may have a very positive outcome. Yet another similarly-situated patient my not respond to the immunotherapeutic for a variety of reasons, only some of which are . Likewise, hormone therapy is only applicable to certain types of cancers and not everyone responds in the same way. Targeted therapies may also be very successful, but a cancer or tumor must be situated such that it can be successfully targeted. These newer therapies, which often receive a lot of very positive press, can create high expectations and hopes (e.g. of avoiding nausea and hair loss, not being sick, maintaining their schedule, etc.) in cancer patients considered for them. Unfortunately, they can result in a highly negative emotional impact on a patient who does not qualify for the treatment (e.g. genetically), or for whom they do not work. This can cause a loss of time in starting more aggressive but less desirable treatments options, as well as have a negative impact on the outcome for patients who lose that hope.

Mushrooms have been used for medicinal and therapeutic purposes for centuries throughout Asia (e.g. in traditional Chinese and Japanese medicine) and around the world. Among the multitude of potentially therapeutic compounds present in mushrooms are complex sugars and polysaccharides (substituted or not) (e.g. glucans, glycosides, glycopeptides, and glycoproteins), terpenes and/or terpenoids, sterols, peptides, amino acids, and other small and large molecules.

Various mushrooms are considered to have anti-cancer, antioxidant, antitumor, antiviral, antibacterial, anti-diabetic, anti-hypercholesterolemic, antiarthritic, anti-asthmatic, anti-obesity, anti-allergenic, anti-thrombotic, anti-inflammatory, anti-bacterial, anti-mutagenic, anti-osteoporotic, and anti-aging therapeutic properties. In addition, some mushrooms reportedly are hypoglycemic, hypotensive, hypocholesterolemic, hepatoprotective, immunomodulatory, and/or beneficial to eye health.

Cannabis spp. have also been used medicinally for centuries. Their therapeutic value is the subject of many current studies. The endogenous endocannabinoid system and related endocannabinoid biology was originally believed to be primarily directed to neurological and psychiatric effects of naturally occurring and exogenous cannabinoids. However, cannabinoids are increasingly recognized as having role(s) in both inflammation and cancer. A role for endocannabinoids in cancer processes has been has been suggested by studying exogenous cannabinoids, particularly naturally occurring cannabinoids from Cannabis sativa, as well as synthetic compounds that can interact with CB1 and CB2 receptors. In vitro and in vivo studies have shown that endocannabinoids inhibit proliferation of cancer cells.

The ongoing discovery of less abundant cannabinoids continues to broaden our knowledge about the range of cannabinoids in plants, and their ability to function in regulation of the endocannabinoid system in humans. Our understanding of the role and potential therapeutic value of such compounds is ongoing. Thus, the use of exogenous cannabinoids and their ability to regulate (particularly upregulate) the endocannabinoid system as a therapeutic approach is being studied.

Other compounds such as terpenes, flavonoids and various botanicals are known to provide beneficial and healthful functions when consumed or administered.

There are ongoing concerns about available treatments, side effects, toxicity, and effectiveness of therapeutics for cancer. And the economic and emotional impact of these conditions is enormous on the personal level for those directly impacted by cancer, as well as for their families, and on the societal level.

There is an ongoing need for new treatment compositions and protocols that are useful for cancers and which provide significant new features and benefits.

### SUMMARY

In a first of the several aspects of this disclosure, the inventor has discovered that certain pharmaceutical and/or nutraceutical compositions generally comprising combinations of one or more edible or medicinal mushrooms or an extract, fraction, or isolate thereof, one or more cannabinoids, one or more terpenes, and optionally, one or more flavonoids.

The mushroom or mushroom extract present in the compositions is from any edible or medicinal mushroom species. The edible or medicinal mushroom in various embodiments comprises one or more of Agaricus, Auricularia, Clitocybe, Ganoderma, Grifola, Hericium, Lentinus, Leucopaxillus, Phellinus, Pleurotus, Sarcodona, and Trametes. In other embodiments, the edible or medicinal mushroom comprises one or more of Albatrellus, Antrodia, Calvatia, Cordyceps, Flammulina, Fomes, Funlia, Inocybe, Inonotus, Lactarius, Russula, Schizophyllum, Suillus, or Xerocomus.

The cannabinoids can be derived from any Cannabis spp. The terpenes and flavonoids can be derived from Cannabis or from any natural or synthetic source. The compositions optionally include other nutraceutical or botanical compounds to provide additional functionality or support.

The inventor has surprisingly discovered that a composition comprising one or more edible or medicinal mushrooms, one or more cannabinoids, one or more terpenes, and at least one flavonoid with has many benefits for cancer treatment, and the compositions provide certain useful properties, such as effectiveness including over a long period of time, low toxicity, no serious side effects, and good tolerance on the part of a wide range of subjects.

In another of its several aspects, provided are methods of treating a subject suffering from cancer. The methods generally comprise administering a therapeutically effective dose of a composition to the subject. The compositions generally comprise one or more edible or medicinal mushrooms or an extract, fraction, or isolate thereof, one or more cannabinoids, one or more terpenes, and optionally, one or more flavonoids. Additional components can also be included, such as S-adenosylmethionine, methylfolate, omega-3 fatty acids, or a B vitamin, vitamin D or a compound providing a biologically-available form thereof. The methods are generally applicable to any type of cancer. Generally, the cancer comprises a common type of cancer such as bladder cancer, brain tumors, breast cancer, cervical cancer, colorectal cancer, endometrial cancer, esophageal cancer, gastric cancer, head and neck cancers, Kaposi sarcoma, kidney (renal cell) cancer, leukemia, liver cancer, lung cancer, lymphoma, melanoma, non-Hodgkin lymphoma, neuroblastoma, ovarian cancer, osteosarcoma and other bone cancers, pancreatic cancer, pituitary tumors, prostate cancer, retinoblastoma, skin cancer, testicular cancer, thyroid cancer, or uterine cancer.

The disclosure also provides a plurality of dosing regimens that utilize the compositions, and variations thereof, on various schedules as dictated by the physiological or psychological health of the subject, and the status of the cancer.

In yet another aspect, methods are provided for optimizing a composition for use in treatment of a subject suffering from cancer. The methods generally employ the use of artificial intelligence algorithms, such as classification algorithms, regression algorithms, clustering algorithms, or a combination thereof.

The methods generally comprise:
a) providing data on the therapeutic effect on the cancer of each of:
   i) a plurality of edible or medicinal mushrooms, mushroom extracts, or components thereof;
   ii) a plurality of cannabinoids or combinations thereof;
   iii) a plurality of terpenes or combinations thereof;
   iv) a plurality of flavonoids or combinations thereof; and optionally,
   v) a plurality of combinations of compositions comprising one or more of mushrooms or extracts thereof, cannabinoids, terpenes, and flavonoid; or
   vi) a plurality of optional ingredients comprising S-adenosylmethionine, methylfolate, omega-3 fatty acids, or a B vitamin or vitamin D or a compound providing a biologically-available form thereof, or combinations thereof;
b) using an artificial intelligence algorithm to analyze the data for the mushrooms or extracts, cannabinoids, terpenes, and flavonoids; and
c) generating one or more base profiles of compositions optimized for therapeutic treatment of the cancer;
d) optionally, using the artificial intelligence algorithm to analyze the data for the combination compositions and the optional ingredients, and
e) generating one or more complete profiles of compositions with and without the optional ingredients.

The cancer generally comprises a prevalent cancer such as bladder cancer, brain tumors, breast cancer, cervical cancer, colorectal cancer, endometrial cancer, esophageal cancer, gastric cancer, head and neck cancers, Kaposi sarcoma, kidney (renal cell) cancer, leukemia, liver cancer, lung cancer, lymphoma, melanoma, non-Hodgkin lymphoma, neuroblastoma, ovarian cancer, osteosarcoma and other bone cancers, pancreatic cancer, pituitary tumors, prostate cancer, retinoblastoma, skin cancer, testicular cancer, thyroid cancer, or uterine cancer.

The data for use in the artificial intelligence algorithm can be obtained from original experiments or literature review.

In a final aspect, this disclosure provides methods for treating cancer in a patient in need thereof. The methods generally comprise the step of administering a composition comprising at least one edible or medicinal mushroom or extract thereof, in combination with at least one cannabinoid, at least one terpene, and at least one flavonoid. The at least one cannabinoid, at least one terpene, and at least one flavonoid are conveniently administered separately from, sequentially to, or simultaneously with the edible or medicinal mushroom. The at least one edible or medicinal mushroom or extract thereof is also administered separately from, sequentially to, or simultaneously with the cannabinoid, terpene, and flavonoid.

These and/or further aspects, features, and advantages of the present invention will become apparent to those skilled in the art in view of this disclosure.

### BRIEF DESCRIPTION OF THE DRAWINGS

### DETAILED DESCRIPTION

Provided herein are compositions and methods for treating cancers including prevalent cancers, but the methods are also applicable to any metastatic or neoplastic disease in a subject. Surprisingly, the compositions allow a modern practitioner to combine the benefits of certain compounds found in edible and medicinal mushrooms (such as used for centuries in Chinese and other traditional medicine practices, and by e.g. herbalists throughout the world), with the positive benefits of another natural substance, cannabis, also used for centuries. The compositions further utilize certain beneficial terpenes and flavonoids derived from Cannabis and other natural or synthetic sources, and other nutraceutical or pharmaceutical compounds. Used properly, these compositions have little risk, few side effects, and are effective for producing measurable and lasting results in patients suffering from cancers or neoplastic diseases.

### Definitions & Abbreviations

Unless expressly defined otherwise, all technical and scientific terms, terms of art, and acronyms used herein have the meanings commonly understood by one of ordinary skill in the art in the field(s) of the invention, or in the field(s) where the term is used. In accordance with this description, the following abbreviations and definitions apply.

The term “cancer” as used herein includes any type of disease characterized by uncontrolled cell growth. Cancer broadly means any type of neoplastic or malignant disease, including metastatic and non-metastatic diseases. Examples of common cancers include breast cancer, colorectal cancer, lung cancer, gastric cancer, bladder cancer, kidney (renal cell) cancer, leukemia, liver cancer, lymphoma, pancreatic cancer, prostate cancer, skin cancer, thyroid cancer, uterine cancer, non-Hodgkin lymphoma, melanoma, endometrial cancer, testicular cancer, ovarian cancer, osteosarcoma and other bone cancers, brain tumors, cervical cancer, esophageal cancer, retinoblastoma, Kaposi sarcoma, head and neck cancers, neuroblastoma, and pituitary tumors.

As the skilled artisan will appreciate, as used herein the term “edible” does not mean merely capable of being eaten. In that overly broad sense, even poisonous or toxic mushrooms are ‘edible’ however lethal or sickening or the like. In contrast “edible mushrooms” is used herein in the sense of mushrooms that are used traditionally or in modern times as sources of food, nutrients, nutraceuticals, flavors, and the like. Edible mushrooms are neither toxic or poisonous as consumed.

“Medicinal mushrooms” as used herein means any mushroom species that has been used traditionally or in modern times as a source of medicinal or therapeutic benefits, healing properties, and / or healthful compounds.

Generally, mushrooms that are edible or medicinal may be grouped together, as there may be many crossovers and it may be difficult to clearly distinguish between the two groups. Examples of edible and / or medicinal mushrooms useful herein include mushrooms of the genera Agaricus, Auricularia, Clitocybe, Ganoderma, Grifola, Hericium, Lentinus, Leucopaxillus, Phellinus, Pleurotus, Sarcodona, and Trametes (aka Coriolus, Polyporus, or Polystictus) are all suitable for use herein. Also useful herein are mushrooms of the genera Albatrellus, Antrodia, Calvatia, Cordyceps, Flammulina, Fomes, Funlia, Inocybe, Inonotus, Lactarius, Russula, Schizophyllum, Suillus, and Xerocomus. Species of particular interest include Agaricus blazei, Albatrellus confluens, Antrodia camphorate, Boletus badius, Clitocybe maxima, Cordyceps militaris, Flanulina velutipes, Fomes fomentarius, Funalia trogii, Ganoderma lucidum, Grifoloa fondosa, Hericium erinaceus, Inocybe umbrinella, Ionatus olbiquus, Lactarius flavidulus, Lentinula edodes, Phellinus linteus, Pleurotus ostreatus, Schizophyllum commune, Suillus placidus, Trametes versicolor, as well as Cordyceps sinensis, C. liangshanensis, C. gunnii, or C. cicadicola.

More generally edible and/or medicinal mushrooms can be useful in connection with the current disclosure for treating cancer, directly or indirectly. For example, the therapeutic uses may include: preventing and/or treating cancer, helping recover from chemotherapy and/or other treatments with significant toxicity, stimulating and / or supporting the immune system, treating primary or secondary infections, or providing antibacterial or antifungal properties, reducing the side effects of radiation therapy, supporting non-chemotherapeutic approaches to treatment, reducing or mitigating the psychological affects (e.g. stress, anxiety, or the like) of a cancer diagnosis, generally promoting of health, providing antioxidant functionality, reducing nausea or stimulating appetite, stimulating or promoting cellular health, or the like.

Specific anticancer or antitumor therapeutic properties include functioning as a reactive oxygen species inducer, a mitotic kinase inhibitor, an anti-mitotic, an angiogenesis inhibitor, a topoisomerase inhibitor, a stimulator of apoptosis, a stimulator of DNA editing and/or repair functions, or as a general immunomodulatory or immunostimulatory compound.

Immune system functions can include stimulating cellular aspect of immunity such as monocytes, natural killer (NK) cells, and dendritic cells. Other potential functions include stimulating T-cell activity, or preventing T-cell apoptosis

Reishi mushrooms have been reported to calm the central nervous system and/or have neuroprotective effect, stimulate the immune system, and act as a prebiotic to support gut health. Reishi has been reported to have a beneficial effect on the adrenals, and to be anxiolytic, reducing anxiety and promoting sleep. Reishi has also been associated with improved memory, and sharpened concentration and focus. Lion’s Mane reportedly calms mental activity and modulates certain neurotransmitters. Cordyceps has been reported to have adaptagenic properties and stimulate the adrenals glands and modulates the nervous system.

“Cannabis” or “Cannabis spp.” as used herein refers to any plant of the genus Cannabis, including plants that may be classified as Cannabis sativa, Cannabis indica, or Cannabis ruderalis. It is well-known that despite the foregoing list, some experts believe that there are only 2 species, and still others consider that there is only a single species (generally, C. sativa). Whatever nomenclature is used, for purposes of this disclosure, “Cannabis” includes all possible members of the genus, without regard to the species to which they are assigned.

As used herein ‘cannabinoids’ means any of a class of compounds that generally can interact with one or more cannabinoid receptors, including the receptors of the endocannabinoid system, in particular, CB1 and CB2. Cannabinoids include e.g., phytocannabinoids and synthetic cannabinoids. Phytocannabinoids are found in several plant species, especially Cannabis spp. Among the most prevalent and most studied cannabinoids are tetrahydrocannabinol (THC), and cannabidiol (CBD). However, there are at least ~120 known cannabinoids that have been identified in Cannabis within certain classes including the tetrahydrocannabinols, cannabidiols, cannabigerols, cannabinols, cannabichromenes, and cannabinodiol. Other cannabinoids, such as cannabicyclol, cannabieslsoin, cannabitriol are currently classed as ‘miscellaneous’ by some researchers. THC is not only a major cannabinoid in Cannabis spp., it is generally the compound responsible for the psychoactive effects of consuming Cannabis. However, other cannabinoids, such as cannabinol may also be at least mildly psychoactive. Certain other cannabinoids such as CBD may help regulate or attenuate the psychoactive effects of other cannabinoids. For purposes herein, compositions may be created with various ratios of cannabinoids, such as the ratio of CBD to THC or other ratios depending the specific person or the specific condition being treated.

As used herein, “terpenes” means any of the organic compounds commonly known as terpenes or terpenoids. Terpenes are generally aromatic compounds classified as isoprene derivatives. Terpenes suitable for use herein include hemiterpenes, monoterpenes, sesquiterpenes, diterpenes, sesterterpenes, triterpenes, sesquaterpenes, tetraterpenes, polyterpenes, and norisoprenoids. Exemplary terpenes that are particularly useful herein include alpha bisabolol, alpha pinene, beta caryophyllene, beta pinene, borneol, camphor, camphene, caryophyllene oxide, cineole, delta-3 carene, eucalyptol, farnesenes, farnesol, fenchol, fenchone, geraniol, guaiol, humulene, isopulegol, limonene, linalool, menthol, myrcene, nerol, nerolidol, ocimene, pinene, phytol, pulegone, terpinene, terpineol, terpinolene, and valencene.

As used herein, “flavonoids” includes any of the class of polyphenolic molecules containing 15 carbon atoms that are naturally produced in plants and are soluble in water. Also included herein as “flavonoids” are natural or synthetic derivative or analogs thereof that have biological activity. Flavonoids of use herein can generally be divided in to 6 groups of structurally-related compounds: chalcones, flavones, isoflavonoids, flavanones, anthoxanthins and anthocyanins. Also useful are flavanols and catechins, as well as glucosides or other derivatives or analogs of any of the foregoing. The flavonoids are found in most fruits and vegetables, particular colorful ones. They are also prevalent in legumes (including soybeans), grains, green and black teas, as well as red wine.

Flavonoids have numerous functions in plants, and act as important cell messengers. Various flavonoids are believed to provide healthful benefits and functions to humans such as anti-viral, anti-cancer, anti-inflammatory, anti-allergic, and anti-oxidant properties. They may also be cardio-protective, cholesterol-lowering, and anti-atherosclerotic. Natural or synthetic flavonoids from any source may be used herein. Generally natural flavonoids are preferred. Flavonoids isolated from Cannabis, such as cannaflavins A, B, and or C, are of interest in certain applications, as are vitexin, isovitexin, apigenin, kaempferol, quercetin, orientin, and luteolin, as well as the catechins found in Cannabis.

“Traditional Jamaican medicinal plants” means any plant that has been used in traditional or indigenous medicine or herbalism practices in Jamaica or other Caribbean states. The book, “Common Medicinal Plants of Portland, Jamaica” by Thomas and Austin, provides a useful list of a number of such plants. The book was published in its second edition in 2010 by CIEER. For purposes herein, the definition of such traditional Jamaican medicinal plants expressly excludes Cannabis spp.

As used herein, the singular form of a word includes the plural, and vice versa, unless the context clearly dictates otherwise. Thus, the references “a”, “an”, and “the” are generally inclusive of the plurals of the respective terms. For example, reference to “a composition” or “a mushroom extract” includes a plurality of such “compositions” or “mushroom extracts.”

The words “comprise”, “comprises”, and “comprising” are to be interpreted inclusively rather than exclusively. Likewise, the terms “include”, “including” and “or” should all be construed to be inclusive, unless such a construction is clearly prohibited from the context. Further, forms of the terms “comprising” or “including” are intended to include embodiments encompassed by the phrases “consisting essentially of” and “consisting of”. Similarly, the phrase “consisting essentially of” is intended to include embodiments encompassed by the phrase “consisting of”.

Where used herein, ranges are provided in shorthand, so as to avoid having to list and describe each and every value within the range. Any appropriate value within the range can be selected, where appropriate, as the upper value, lower value, or the terminus of the range.

The methods and devices and/or other advances disclosed here are not limited to particular methodology, protocols, and/or structures described herein because, as the skilled artisan will appreciate, they may vary. Further, the terminology used herein is for the purpose of describing particular embodiments only, and is not intended to, and does not, limit the scope of that which is disclosed or claimed.

Although any devices, methods, articles of manufacture, or other means or materials similar or equivalent to those described herein can be used in the practice of the present invention, the preferred compositions, methods, articles of manufacture, or other means or materials are described herein.

All patents, patent applications, publications, technical and/or scholarly articles, and other references cited or referred to herein are in their entirety incorporated herein by reference to the extent permitted under applicable law. Any discussion of those references is intended merely to summarize the assertions made therein. No admission is made that any such patents, patent applications, publications or references are prior art, or that any portion thereof is either relevant or material to the patentability of what is claimed herein. Applicant specifically reserves the right to challenge the accuracy and pertinence of any assertion that such patents, patent applications, publications, and other references are prior art, or are relevant, and/or material.

### Abbreviations

The following abbreviations apply unless indicated otherwise:

| | |
|---|---|
| APM: | “Apollon Medical” strain of C. sativa |
| CBD: | cannabidiol; |
| CBG: | cannabigerol; |
| CBN: | cannabinol; |
| DMT: | N,N-Dimethyltryptamine; |
| DOI: | 1-(2,5-dimethoxy-4-iodophenyl-2-aminopropane; |
| 5-HT: | 5-hydroxytryptamine; |
| KNN: | K Nearest Neighbor; |
| LSD: | lysergic acid diethylamide; |
| NK: | natural killer cells; |
| OCD: | obsessive compulsive disorder; |
| SVM: | Support Vector Machines; and |
| THC: | tetrahydrocannabinol. |

### Detailed Description of Illustrative Embodiments

In a first of its several aspects, compositions generally comprising one or more edible or medicinal mushrooms or extracts, fractions, isolates, or components thereof are provided. The compositions further generally comprise one or more cannabinoids, one or more terpenes, and optionally, one or more flavonoids.

In various embodiments, the edible or medicinal mushroom(s) comprise one or more of Agaricus, Auricularia, Clitocybe, Ganoderma, Grifola, Hericium, Lentinus, Leucopaxillus, Phellinus, Pleurotus, Sarcodona, and Trametes. In other embodiments, the edible or medicinal mushroom(s) comprises one or more of Albatrellus, Antrodia, Calvatia, Cordyceps, Flammulina, Fomes, Funlia, Inocybe, Inonotus, Lactarius, Russula, Schizophyllum, Suillus, or Xerocomus.

These mushrooms are all well-known edible and / or medicinal mushrooms with a long history of use. Without limiting the invention to any particular theory of operation, mushrooms, such as Turkey Tail, Reishi, shitake, maitake, and many others have a plethora of beneficial and potentially therapeutic compounds present in them. For example, the polysaccharide content in Reishi mushroom, particularly the beta-1,3 D-glucan, has been shown to up-regulate the production of certain lymphocytes, T-helper cells, T-killer cells, and macrophages. The same glycan has also been shown to be involved with suppressing tumor necrosis factor (TNF-α). Despite the name, the cytokine TNF has shown in certain cancers to actual interfere with processes that would otherwise fight cancer or eliminate cancer cells.

Regardless of the mechanism(s) responsible, the compositions preferably comprise the benefits of such edible or medicinal mushroom(s) as described above.

The mushroom(s) in certain presently preferred embodiments comprises one or more species such as Agaricus blazei, Albatrellus confluens, Antrodia camphorate, Boletus badius, Clitocybe maxima, Cordyceps militaris, Cordyceps sinensis, Cordyceps liangshanensis, Cordyceps gunnii, Cordyceps cicadicola, Flanulina velutipes, Fomes fomentarius, Funalia trogii, Ganoderma lucidum, Grifoloa fondosa, Hericium erinaceus, Inocybe umbrinella, Ionatus olbiquus, Lactarius flavidulus, Lentinula edodes, Phellinus linteus, Pleurotus ostreatus, Schizophyllum commune, Suillus placidus, or Trametes versicolor.

The edible or medicinal mushrooms or extracts can be present as fresh mushrooms, or as a dried mushroom or extract thereof, a lyophilized mushroom preparation, a mushroom powder, or an aqueous or alcohol extract (e.g. an ethanolic or other alcohol extract) of mushroom. Extracts for purposes herein can include hot- or cold-water extracts. Mushroom concentrates, or partially purified or even extensively purified mushroom fractions, or fully purified components or isolates from a mushroom are also useful herein.

In certain embodiments, preferably, various extracts, concentrates, or partially purified fractions, or the like, are enriched for one or more advantageous mushroom component, or compounds with desirable effects on treatment of cancer, immune status, or general health. In one embodiment, the extract, concentrate, partially purified fraction, or the like are enriched for one or more phenolic acids, flavonoids, triterpenes, carotenoids, stilbenes, sterols, fatty acids, lignans, chitosan, polyphenols, polysaccharides, glycosides, glucans, polysaccharide-protein complexes, glycoproteins, polysaccharopeptide, krestin, tocopherols, peptides, cyclic peptides, or other amino compounds found in the mushroom.

While certain edible and / or medicinal mushrooms have been explored for their use in the therapeutic treatment of cancer, the inventors have discovered that by providing mushroom extracts with additional compounds such as cannabinoids, terpenes, flavonoids, and other components, anticancer benefits can be derived from the treatments with better outcomes and fewer side effects.

The compositions in various embodiments therefor also include cannabinoids, comprising one or more of cannabidiol (CBD), cannabinol (CBN), cannabigerol (CBG), or tetrahydrocannabinol (THC).

In certain embodiments, the cannabinoids are derived from hemp, or the THC content is removed from the cannabinoids such that THC is present in the composition at less than about 0.3 percent. In various embodiments, the THC content may be less than 0.2, or even less than 0.1% of the composition.

In other embodiments, the ratio of cannabinoids is set in the compositions. For example, in certain embodiments, the ratio of CBD to THC ranges from about 1:5 to about 30:1 or more. In other embodiments the ration may range from about 1:2 to about 10:1. In still other embodiments, the ratio of CBD to THC may be about 1:1 to 5:1. In other cases, the ration of e.g. CBD to CBG or CBN may be set based on any of a variety of factors, including the health status of the subject being treated, the symptoms of the subject, the condition(s) being treated, and or one or more physiological or genetic criteria.

In certain embodiments the compositions may be provided in completely customized or personalized formulations for each person being treated — i.e. as personalized medicines. In such cases, the compositions may be adjusted based on initial or subsequent blood work, enzyme test results, bioinformatic data (including measurements of e.g. the genome, transcriptome, proteome, metabolome, or any portion thereof, for a subject), the type and stage of cancer, specific markers, antigen, or receptors of the cancer, specific symptomology, or the like. The formulation of the compositions may also be changed based on the results from an initial treatment, subsequent treatment, or based on subsequent tests.

In various presently preferred embodiments, the cannabinoids are derived from Cannabis spp. or an extract thereof. Cannabis can generally be concentrated or extracted (e.g. via mechanical or chemical means) to obtain cannabinoids. Mechanical means of extracting oils from plants, such as pressing, have been used for centuries, and may be suitable for use herein. Extraction via chemical means includes extraction with various volatile solvents that range from hydrocarbon solvents such as butane, hexanes or propane, to supercritical fluids, alcohol (e.g. isopropanol, butanol, or ethanol), steam, or even water. Two very common methods are extraction with supercritical carbon dioxide, or ethanol, both of which are particularly useful herein. Extracts can be also be distilled e.g. to remove additional compounds of interest, or to concentrate them. Certain components can be removed, e.g. by treatment with steam to strip certain volatiles, which can be captured as an additional component from the Cannabis.

In various embodiments, cannabinoids present in an ethanolic extract or supercritical CO, extract of Cannabis sativa are preferred for use herein. The extract comprises one or more of Ringo’s Gift, Harle Tsu, ACDC, Charlotte’s Web, The Gift, or Pineberry strains of Cannabis sativa. “Apollon Medical”(“APM”), a proprietary strain commercially available from Apollon Formularies, is also useful herein.

The composition in various embodiments include terpenes comprising one or more monoterpenes, one or more sesquiterpenes, or a combination thereof. The terpenes comprise one or more of alpha bisabolol, alpha pinene, beta caryophyllene, beta pinene, borneol, camphor, camphene, caryophyllene oxide, cineole, delta-3 carene, eucalyptol, farnesenes, farnesol, fenchol, fenchone, geraniol, guaiol, humulene, isopulegol, limonene, linalool, menthol, myrcene, nerol, nerolidol, ocimene, pinene, phytol, pulegone, terpinene, terpineol, terpinolene, or valencene.

In one embodiment, the terpenes comprise phytol, limonene, humulene, myrcene, phellandrene, caryophyllene, linalool, pinene, or a combination thereof. In other embodiments, the terpenes preferably comprise one or more of limonene, myrcene, beta-caryophyllene, linalool, alpha pinene, or a combination thereof.

In presently preferred embodiments, the terpenes are derived from Cannabis spp. or an extract thereof. The terpenes can be derived from any source and in certain embodiments, they can be present in steam distillate or an ethanolic extract of Cannabis sativa.

The compositions in certain embodiments include extracts of Cannabis spp, such as C. sativa as a source of cannabinoids and/or terpenes. In various embodiments, the C. sativa comprises one or more of Ringo’s Gift, Harle Tsu, ACDC, Charlotte’s Web, The Gift, or Pineberry strains. In other embodiments, the C. sativa comprises the proprietary Apollon Formularies strain, APM.

In various other embodiments, the compositions may further comprise one or more flavonoids. The flavonoids can comprise chalcones, flavones, isoflavonoids, flavanones, anthoxanthins, anthocyanins, flavonols, or glucosides or other biologically active derivatives or analogs thereof, and combinations of any number of the foregoing. In certain embodiments, flavonoids are included in the compositions are isolated or derived from a plant, mushroom, or other natural source.

In another embodiment, the composition still further comprises an extract or fraction from one or more traditional Jamaican medicinal plants other than Cannabis spp. Any of the traditional Jamaican or Carribbean medicinal plants may be useful herein. In one embodiment, the traditional medicinal plants comprise guinea hen weed (Petiveria alliacea), and / or soursop (Annona murata). In a presently preferred embodiments, the compositions include a whole plant extract, or an extract from any parts or portion thereof including but not limited to leaves, stems, flowers, roots, fruit, seeds, or the like.

In terms of compounding the compositions, the skilled artisan will appreciate that methods of maximizing the efficacy of the composition such as by enhancing the bioavailability of one or more components, or by providing the components in optimized ratios, for example one component to another with which it interacts, or each component to the others in ratio(s) that optimize the absorption into the gut or bloodstream, or enhance the therapeutic effect of the composition. The skilled artisan will also understand that some information useful in improving the compounding may be obtained empirically.

In various embodiments, the compositions, or one or more components thereof, may be solubilized, micronized, provided as, for example. extracts, powders, lyophilized powders, concentrates, tinctures, essential oils, aqueous or lipid suspensions, emulsions, microemulsions, or nano-emulsions, or in whole or part as liposomal, vesicular, or other delivery systems. As described below, the compounding or formulation of any of the compositions provided herein may be optimized for the intended delivery route.

The compositions may be administered and delivered as pharmaceuticals, however, it is also contemplated that one or more of the compositions may be formulated for administration and delivery by oral routes that include as food and beverages, including solid, semisolid, and liquid foods, such as smoothies, shakes, pudding, broths, teas, and soups. The food and or beverage compositions can also include hot, cold, or even frozen foods (such as frozen desserts).

In another aspect of the disclosure, provided are methods of treating a subject suffering from cancer. The methods generally comprise administering a therapeutically effective dose of a composition to the subject. The composition typically comprises one or more edible or medicinal mushrooms or an extract, fraction, or isolate thereof, one or more cannabinoids, one or more terpenes, and optionally, one or more flavonoids.

The cancer in various embodiments is a common cancer such as bladder cancer, brain tumors, breast cancer, cervical cancer, colorectal cancer, endometrial cancer, esophageal cancer, gastric cancer, head and neck cancers, Kaposi sarcoma, kidney (renal cell) cancer, leukemia, liver cancer, lung cancer, lymphoma, melanoma, non-Hodgkin lymphoma, neuroblastoma, ovarian cancer, osteosarcoma and other bone cancers, pancreatic cancer, pituitary tumors, prostate cancer, retinoblastoma, skin cancer, testicular cancer, thyroid cancer, or uterine cancer.

The composition for use in the methods is generally as described above for the first aspect. The description of the compositions above is incorporated by reference herein for purposes of the present methods.

In various embodiments, the compositions for use in the methods further comprise one or more optional ingredients comprising S-adenosylmethionine, methylfolate, omega-3 fatty acids, or a B vitamin, vitamin D or a compound providing a biologically-available form thereof.

The terpenes for use with the compositions can comprise one or more monoterpenes, one or more sesquiterpenes, or a combination thereof. In various embodiments, the terpenes comprise one or more of alpha bisabolol, alpha pinene, beta caryophyllene, beta pinene, borneol, camphor, camphene, caryophyllene oxide, cineole, delta-3 carene, eucalyptol, farnesenes, farnesol, fenchol, fenchone, geraniol, guaiol, humulene, isopulegol, limonene, linalool, menthol, myrcene, nerol, nerolidol, ocimene, pinene, phytol, pulegone, terpinene, terpineol, terpinolene, or valencene. In certain preferred embodiments, the terpenes are derived from Cannabis spp. or an extract thereof. The terpenes can be present in steam distillate or an ethanolic extract of Cannabis sativa, and can comprise limonene, myrcene, beta-caryophyllene, linalool, alpha pinene, or a combination thereof, in some embodiments. The Cannabis sativa in one embodiment includes one or more of Ringo’s Gift, Harle Tsu, ACDC, Charlotte’s Web, The Gift, or Pineberry strains.

The compositions for use with the methods also comprise one or more flavonoids that can be chalcones, flavones, isoflavonoids, flavanones, anthoxanthins, anthocyanins, flavonols, or glucosides or other biologically active derivatives or analogs thereof, and preferably they are from a plant, mushroom, or other natural source.

Presently preferred flavonoids include cannaflavin A, cannaflavin B, or cannaflavin C, vitexin, isovitexin, apigenin, kaempferol, quercetin, orientin, luteolin, a catechin found in Cannabis, or a combination of any of the foregoing.

In certain embodiments, the methods further comprise one or more steps of administering an additional therapeutically effective dose of the composition.

Preferably the additional administering steps are performed on a periodic basis of any frequency or schedule. For example, the administration or dosing can conveniently be on e.g. a daily, thrice weekly, twice weekly, weekly, biweekly, monthly, bimonthly, quarterly, semi-annual, or annual basis. The administration need not be the same over every period of time. By way of nonlimiting example, administration could be daily for a week, then weekly for a month. Or the administration could be every 4 months for a year, then every 6 months thereafter. Similarly, the actual amount of the composition or dosage administration can vary. For example, a monthly dosage schedule could feature a dose of x for the first dosage each quarter, and a dose of 0.1x for the remaining months in each quarter.

Just as the composition can be ‘personalized’, so can the administration or dosing schedule. Thus, in various embodiments, the methods further comprise the step of periodically assessing one or more of the subject’s medication levels, enzyme levels, or other indicators of physiological health or status, genetic markers or antigen presence in the cancer cells, or the like, in order to determine the periodic basis for administration.

The methods provide for administration of the compositions via any useful route, including parenteral (intravenous, intra-arterial, intramuscular, intraperitoneal, or subcutaneous), oral, nasal, ocular, transmucosal (buccal, vaginal, or rectal), transdermal, or via inhalation.

It should be noted that the route of dosing or administration of compositions can vary over the course of treating a subject or patient with multiple steps of treatment, as well as from subject to subject, or with different types of cancer. For example, administration via one route may be useful when administering a larger dose and a different route may be useful for smaller doses. Or, administration via a particular route may be appropriate initially, with subsequent doses conveniently administered through another route.

In one embodiment, the method further comprises a step of providing to the subject additional treatment of the cancer comprising:
i) one or more doses of a chemotherapeutic agent;
ii) one or more treatments with ionizing radiation;
iii) one or more doses of an immunotherapeutic;
iv) one or more targeted treatments of the cancer;
v) one or more other treatments specifically provided to treat the cancer;
or any combination of any of the foregoing.

Methods that further comprise a step of providing to the subject an additional composition are also provided. The additional composition may be administered in between doses of the base compositions, or may be provided on a separate and independent periodic basis. The addition compositions generally comprise any combination of less than three of the following:
i) one or more edible or medical mushrooms or an extract, fraction, or isolate thereof;
ii) one or more cannabinoids;
iii) one or more terpenes; or
iv) one or more flavonoids;

The additional compositions further optionally comprise any combination of one or more of S-adenosylmethionine, methylfolate, omega-3 fatty acids, or a B vitamin, vitamin D, or a compound providing a biologically-available form thereof.

In various embodiments of the methods:
i) the one or more edible or medical mushrooms comprise Agaricus, Auricularia, Clitocybe, Ganoderma, Grifola, Hericium, Lentinus, Leucopaxillus, Phellinus, Pleurotus, Sarcodona, Trametes, Albatrellus, Antrodia, Calvatia, Cordyceps, Flammulina, Fomes, Funlia, Inocybe, Inonotus, Lactarius, Russula, Schizophyllum, Suillus, or Xerocomus;
ii) the cannabinoids comprise one or more of cannabidiol (CBD), cannabinol (CBN), cannabigerol (CBG), or tetrahydrocannabinol (THC);
iii) the terpenes comprise one or more of alpha bisabolol, alpha pinene, beta caryophyllene, beta pinene, borneol, camphor, camphene, caryophyllene oxide, cineole, delta-3 carene, eucalyptol, farnesenes, farnesol, fenchol, fenchone, geraniol, guaiol, humulene, isopulegol, limonene, linalool, menthol, myrcene, nerol, nerolidol, ocimene, pinene, phytol, pulegone, terpinene, terpineol, terpinolene, or valencene; and
iv) the one or more flavonoids comprise chalcones, flavones, isoflavonoids, flavanones, anthoxanthins, anthocyanins, flavonols, or glucosides or other biologically active derivatives or analogs thereof.

The cancer can comprise any metastatic or neoplastic disease such as bladder cancer, breast cancer, colorectal cancer, endometrial, kidney (renal) cancer, leukemia, lung cancer, non-Hodgkin’s lymphoma, pancreatic cancer, prostate cancer, skin cancers, stomach cancer, or thyroid cancer.

In one embodiment of the methods, the composition further comprises an extract or fraction from one or more traditional Jamaican medicinal plants other than Cannabis spp. The traditional medicinal plants comprise guinea hen weed (Petiveria alliacea), or soursop (Annona murata) in certain embodiments.

The skilled artisan will appreciate that the methods are flexible as set forth herein, an aspect which is particularly useful given the varied and nature of the psychological disorders which they are intended to treat.

In yet another aspect of the disclosure, methods of optimizing a composition for use in treatment of a subject suffering from cancer using artificial intelligence are provided herein. The methods generally comprise, for each cancer of interest, or for a subject in need of therapeutic compositions for such cancer:
a) providing data on the therapeutic effect on the cancer of each of:
   i) a plurality of edible or medicinal mushrooms, mushroom extracts, or components thereof;
   ii) a plurality of cannabinoids or combinations thereof;
   iii) a plurality of terpenes or combinations thereof;
   iv) a plurality of flavonoids or combinations thereof; and optionally,
   v) a plurality of combinations of compositions comprising one or more of mushrooms or extracts thereof, cannabinoids, terpenes, and flavonoid; or
   vi) a plurality of optional ingredients comprising S-adenosylmethionine, methylfolate, omega-3 fatty acids, or a B vitamin or vitamin D or a compound providing a biologically-available form thereof, or combinations thereof;
b) using an artificial intelligence algorithm to analyze the data for the mushrooms or extracts, cannabinoids, terpenes, and flavonoids; and
c) generating one or more base profiles of compositions optimized for therapeutic treatment of the cancer;
d) optionally, using the artificial intelligence algorithm to analyze the data for the combination compositions and the optional ingredients, and
e) generating one or more complete profiles of compositions with and without the optional ingredients.

In certain embodiments, the cancer comprises bladder cancer, brain tumors, breast cancer, cervical cancer, colorectal cancer, endometrial cancer, esophageal cancer, gastric cancer, head and neck cancers, Kaposi sarcoma, kidney (renal cell) cancer, leukemia, liver cancer, lung cancer, lymphoma, melanoma, non-Hodgkin lymphoma, neuroblastoma, ovarian cancer, osteosarcoma and other bone cancers, pancreatic cancer, pituitary tumors, prostate cancer, retinoblastoma, skin cancer, testicular cancer, thyroid cancer, or uterine cancer. Generally, the data for the method are obtained from original experiments and/or reviews of the relevant scientific literature.

The artificial intelligence algorithm can comprise any useful software or algorithm approach capable of making the distinctions required. In various embodiments, the algorithm comprises a classification algorithm, a regression algorithm, a clustering algorithm, or a combination thereof.

In one embodiment, the methods comprise a classification algorithm that is a naïve Bayes algorithm, decision tree, random forest algorithm, Support Vector Machines, or K Nearest Neighbor algorithm.

In another embodiment, the methods comprise a regression algorithm that is a liner regression, lasso regression, logistic regression, or multivariate regression.

In yet another embodiment, the methods comprise a clustering algorithm that is a K-means clustering, fuzzy C-means algorithm, expectation-maximization algorithm, or hierarchical clustering algorithm.

The skilled artisan will appreciate that the methods are designed to optimize the compositions, and that such optimization as set forth above can be with respect to each particular cancer or even each particular variant of a cancer. However, the compositions can also be optimized for, and a profile of relevant compositions generated for each particular subject, e.g. for a ‘personalized medicine’ approach.

Thus, also provided herein are the methods comprising the additional step of providing subject-specific data comprising, e.g. initial or subsequent blood work, enzyme test results, bioinformatic data (including measurements of e.g. the genome, transcriptome, proteome, metabolome, or any portion thereof, for a subject), specific symptomology, or the like. The artificial intelligence algorithm is then used to further optimize the composition based on those data in addition to the disorder-specific data. The optimized formulation of the compositions may also be changed based on data from the results from an initial treatment, subsequent treatment, or based on subsequent tests of the subject.

In certain embodiments, the cancer comprises a prevalent form of cancer such as bladder cancer, breast cancer, colorectal cancer, endometrial, kidney (renal) cancer, leukemia, lung cancer, non-Hodgkin’s lymphoma, pancreatic cancer, prostate cancer, skin cancers, stomach cancer, or thyroid cancer.

The plurality of edible or medicinal mushrooms, mushroom extracts, or components for which data are analyzed generally comprise one or more of the species Agaricus blazei, Albatrellus confluens, Antrodia camphorate, Boletus badius, Clitocybe maxima, Cordyceps militaris, Flanulina velutipes, Fomes fomentarius, Funalia trogii, Ganoderma lucidum, Grifoloa fondosa, Hericium erinaceus, Inocybe umbrinella, Ionatus olbiquus, Lactarius flavidulus, Lentinula edodes, Phellinus linteus, Pleurotus ostreatus, Schizophyllum commune, Suillus placidus, Trametes versicolor, Cordyceps sinensis, C. liangshanensis, C. gunnii, or C. cicadicola.

A further aspect of the invention provides methods for the treatment of cancer. The methods generally comprise the step of administering to a patient in need thereof a composition comprising at least one edible or medicinal mushroom or extract thereof, in combination with at least one cannabinoid, at least one terpene, and at least one flavonoid. In one embodiment the at least one cannabinoid, at least one terpene, and at least one flavonoid are administered separately from, sequentially to, or simultaneously with the edible or medicinal mushroom or extract thereof. In another embodiment, the at least one edible or medicinal mushroom or extract thereof is also administered separately from, sequentially to, or simultaneously with the cannabinoid, terpene, and flavonoid.

Again, the composition with respect to this aspect of the disclosure can comprise any of the compositions described hereinabove. In one embodiment of the methods:
i) the at least one edible or medicinal mushroom or extract comprises comprise Agaricus blazei, Albatrellus confluens, Antrodia camphorate, Boletus badius, Clitocybe maxima, Cordyceps militaris, Flanulina velutipes, Fomes fomentarius, Funalia trogii, Ganoderma lucidum, Grifoloa fondosa, Hericium erinaceus, Inocybe umbrinella, Ionatus olbiquus, Lactarius flavidulus, Lentinula edodes, Phellinus linteus, Pleurotus ostreatus, Schizophyllum commune, Suillus placidus, Trametes versicolor, Cordyceps sinensis, C. liangshanensis, C. gunnii, or C. cicadicola;
ii) the at least one cannabinoid comprises CBD, CBG, CBN, and THC extracted from Cannabis sativa Ringo’s Gift strain;
iii) the at least one terpene comprises limonene, myrcene, beta-caryophyllene, linalool, alpha pinene, or a combination thereof; and
iv) the at least one flavonoid comprises a chalcone, flavone, isoflavonoid, flavanone, anthoxanthin, anthocyanin, flavonol, or glucoside or other biologically active derivatives or analogs thereof.

In various embodiments of the methods, one or more of the components have one more of the following functions:
i) induces apoptosis of cancer cells;
ii) inhibits the VEGF pathway and/or prevent angiogenesis of cancer cells;
iii) disrupts one or more aspect of cell growth of cancer cells;
iv) restores normal differentiation of cancer cells, or restores normal cell cycle in cancer cells; or
v) inhibits one of more of migration, adhesion, or invasion of cancer cells.

In yet another embodiment of the methods provided in this aspect of the disclosure, the composition further comprises an extract or fraction from one or more traditional Jamaican medicinal plants other than Cannabis spp. Traditional medicinal plants comprise guinea hen weed (Petiveria alliacea), or soursop (Annona murata) are contemplated as useful herein.

The scope of the invention is set forth in the claims appended hereto, subject, for example, to the limits of language. Although specific terms are employed to describe the invention, those terms are used in a generic and descriptive sense and not for purposes of limitation. Moreover, while certain presently preferred embodiments of the claimed invention have been described herein, those skilled in the art will appreciate that such embodiments are provided by way of example only. In view of the teachings provided herein, certain variations, modifications, and substitutions will occur to those skilled in the art. It is therefore to be understood that the invention may be practiced otherwise than as specifically described, and such ways of practicing the invention are either within the scope of the claims, or equivalent to that which is claimed, and do not depart from the scope and spirit of the invention as claimed.

What is claimed is:

1. A composition comprising one or more edible or medicinal mushrooms or an extract, fraction, or isolate thereof, one or more cannabinoids, one or more terpenes, and optionally, one or more flavonoids.

2. (canceled)

3. (canceled)

4. The composition of claim 1 wherein the edible or medicinal mushroom comprises one or more of Agaricus blazei, Albatrellus confluens, Antrodia camphorate, Boletus badius, Clitocybe maxima, Cordyceps militaris, Cordyceps sinensis, Cordyceps liangshanensis, Cordyceps gunnii, Cordyceps cicadicola, Flanulina velutipes, Fomes fomentarius, Funalia trogii, Ganoderma lucidum, Grifoloa fondosa, Hericium erinaceus, Inocybe umbrinella, Ionatus olbiquus, Lactarius flavidulus, Lentinula edodes, Phellinus linteus, Pleurotus ostreatus, Schizophyllum commune, Suillus placidus, or Trametes versicolor.

5. The composition of claim 1 wherein the edible or medicinal mushroom comprises fresh mushroom, dried mushroom, lyophilized mushroom, a mushroom powder, an aqueous or ethanolic extract of mushroom, or concentrate thereof, or a partially purified or purified mushroom fraction or component, or an isolate thereof.

6. The composition of claim 5 wherein the aqueous or ethanolic extract of mushroom, or concentrate thereof, or the partially purified or purified mushroom fraction or component, or isolate thereof are enriched for one or more of phenolic acids, flavonoids, triterpenes, carotenoids, stilbenes, sterols, fatty acids, lignans, chitosan, polyphenols, polysaccharides, glycosides, glucans, polysaccharide-protein complexes, glycoproteins, polysaccharopeptide, krestin, tocopherols, peptides, cyclic peptides, or amino compounds found in the mushroom.

7. The composition of claim 1 wherein the cannabinoids comprise one or more of cannabidiol (CBD), cannabinol (CBN), cannabigerol (CBG), or tetrahydrocannabinol (THC);
wherein the THC is present at less than about 0.3 percent or wherein the ratio of CBD to THC is about 1:5 to about 30:1 or more.

8. (canceled)

9. (canceled)

10. (canceled)

11. The composition of claim 7 wherein the cannabinoids or the terpenes are present in a steam distillate or ethanolic extract of Cannabis sativa comprising one or more of Ringo’s Gift, Harle Tsu, ACDC, Charlotte’s Web, The Gift, or Pineberry strains.

12. (canceled)

13. (canceled)

14. The composition of claim 11 wherein the terpenes comprise one or more of alpha bisabolol, alpha pinene, beta caryophyllene, beta pinene, borneol, camphor, camphene, caryophyllene oxide, cineole, delta-3 carene, eucalyptol, farnesenes, farnesol, fenchol, fenchone, geraniol, guaiol, humulene, isopulegol, limonene, linalool, menthol, myrcene, nerol, nerolidol, ocimene, phellandrene, pinene, phytol, pulegone, terpinene, terpineol, terpinolene, or valencene.

15. (canceled)

16. (canceled)

17. (canceled)

18. (canceled)

19. The composition of claim 1 wherein the one or more flavonoids comprise chalcones, flavones, isoflavonoids, flavanones, anthoxanthins, anthocyanins, flavonols, or glucosides or other biologically active derivatives or analogs thereof.

20. (canceled)

21. The composition of claim 19 wherein the one or more flavonoids comprise cannaflavin A, cannaflavin B, or cannaflavin C, vitexin, isovitexin, apigenin, kaempferol, quercetin, orientin, luteolin, a catechin found in Cannabis, or a combination of any of the foregoing.

22. The composition of claim 1 further comprising an extract or fraction from one or more traditional Jamaican medicinal plants other than Cannabis spp.

23. (canceled)

24. The composition of claim 1 further comprising one or more optional ingredients comprising S-adenosylmethionine, methylfolate, omega-3 fatty acids, or a B vitamin, vitamin D, or a compound providing a biologically-available form thereof.

25. A method of treating a subject suffering from cancer which comprises administering to the subject a therapeutically effective dose of a composition comprising one or more edible or medicinal mushrooms or an extract, fraction, or isolate thereof, one or more cannabinoids, one or more terpenes, and optionally, one or more flavonoids;
wherein the cancer comprises bladder cancer, brain tumors, breast cancer, cervical cancer, colorectal cancer, endometrial cancer, esophageal cancer, gastric cancer, head and neck cancers, Kaposi sarcoma, kidney (renal cell) cancer, leukemia, liver cancer, lung cancer, lymphoma, melanoma, non-Hodgkin lymphoma, neuroblastoma, ovarian cancer, osteosarcoma and other bone cancers, pancreatic cancer, pituitary tumors, prostate cancer, retinoblastoma, skin cancer, testicular cancer, thyroid cancer, or uterine cancer.

26. The method of claim 25 wherein the composition further comprises one or more optional ingredients comprising S-adenosylmethionine, methylfolate, omega-3 fatty acids, or a B vitamin, vitamin D or a compound providing a biologically-available form thereof.

27. (canceled)

28. The method of claim 26 wherein the edible or medicinal mushroom comprises one or more of Agaricus blazei, Albatrellus confluens, Antrodia camphorate, Boletus badius, Clitocybe maxima, Cordyceps militaris, Flanulina velutipes, Fomes fomentarius, Funalia trogii, Ganoderma lucidum, Grifoloa fondosa, Hericium erinaceus, Inocybe umbrinella, Ionatus olbiquus, Lactarius flavidulus, Lentinula edodes, Phellinus linteus, Pleurotus ostreatus, Schizophyllum commune, Suillus placidus, Trametes versicolor, Cordyceps sinensis, C. liangshanensis, C. gunnii, or C. cicadicola.

29. The method of claim 26 wherein the edible or medicinal mushroom comprises fresh mushroom, dried mushroom, lyophilized mushroom, a mushroom powder, an aqueous or ethanolic extract of mushroom, or concentrate thereof, or a partially purified or purified mushroom fraction or component, or an isolate thereof, said mushroom enriched for one or more of phenolic acids, flavonoids, triterpenes, carotenoids, stilbenes, sterols, fatty acids, lignans, chitosan, polyphenols, polysaccharides, glycosides, glucans, polysaccharide-protein complexes, glycoproteins, polysaccharopeptide, krestin, tocopherols, peptides, cyclic peptides, or amino compounds found in the mushroom.

30. (canceled)

31. The method of claim 29 wherein the cannabinoids comprise one or more of cannabidiol (CBD), cannabinol (CBN), cannabigerol (CBG), or tetrahydrocannabinol (THC);
wherein the THC is present at less than about 0.3 percent or wherein the ratio of CBD to THC is about 1:5 to about 30:1 or more.

32. (canceled)

33. (canceled)

34. The method of claim 25 wherein the cannabinoids or the terpenes are derived from Cannabis spp. or an extract or distillate thereof; wherein the Cannabis spp. comprises one or more of Ringo’s Gift, Harle Tsu, ACDC, Charlotte’s Web, The Gift, or Pineberry strains of Cannabis sativa.

35. (canceled)

36. (canceled)

37. (canceled)

38. The method of claim 34 wherein the terpenes comprise one or more of alpha bisabolol, alpha pinene, beta caryophyllene, beta pinene, borneol, camphor, camphene, caryophyllene oxide, cineole, delta-3 carene, eucalyptol, farnesenes, farnesol, fenchol, fenchone, geraniol, guaiol, humulene, isopulegol, limonene, linalool, menthol, myrcene, nerol, nerolidol, ocimene, pinene, phytol, pulegone, terpinene, terpineol, terpinolene, or valencene.

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. The method of claim 25 further comprising a step of providing to the subject additional treatment of the cancer comprising:
one or more doses of a chemotherapeutic agent;
one or more treatments with ionizing radiation;
one or more doses of an immunotherapeutic;
one or more targeted treatments of the cancer;
one or more other treatments specifically provided to treat the cancer; or any combination of any of the foregoing.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. A method for the treatment of cancer comprising the step of administering to a patient in need thereof a composition comprising at least one edible or medicinal mushroom or extract thereof, in combination with at least one cannabinoid, at least one terpene, and at least one flavonoid; wherein the at least one cannabinoid, at least one terpene, and at least one flavonoid is administered separately from, sequentially to, or simultaneously with the edible or medicinal mushroom; and wherein the at least one edible or medicinal mushroom or extract thereof is also administered separately from, sequentially to, or simultaneously with the cannabinoid, terpene, and flavonoid, wherein:
i) the at least one edible or medicinal mushroom or extract comprises comprise Agaricus blazei, Albatrellus confluens, Antrodia camphorate, Boletus badius, Clitocybe maxima, Cordyceps militaris, Flanulina velutipes, Fomes fomentarius, Funalia trogii, Ganoderma lucidum, Grifoloa fondosa, Hericium erinaceus, Inocybe umbrinella, Ionatus olbiquus, Lactarius flavidulus, Lentinula edodes, Phellinus linteus, Pleurotus ostreatus, Schizophyllum commune, Suillus placidus, Trametes versicolor, Cordyceps sinensis, C. liangshanensis, C. gunnii, or C. cicadicola;
ii) the at least one cannabinoid comprises CBD, CBG, CBN, and THC extracted from Cannabis sativa Ringo’s Gift strain;
iii) the at least one terpene comprises limonene, myrcene, beta-caryophyllene, linalool, alpha pinene, or a combination thereof; and
iv) the at least one flavonoid comprises a chalcone, flavone, isoflavonoid, flavanone, anthoxanthin, anthocyanin, flavonol, or glucoside or other biologically active derivatives or analogs thereof.

65. (canceled)

66. (canceled)

67. (canceled)
